(19) United States
(12) Patent Application Publication
(10) Pub. No.: US 2010/0184785 A1
(43) Pub. Date: Jul. 22, 2010

(54) PHARMACEUTICAL FORMULATION FOR THE PRODUCTION OF CHEWABLE TABLETS AND LOZENGES

(75) Inventors: Karl Kolter, Limburgerhof (DE); Michael Schönherr, Frankenthal (DE); Silke Gebert, Grunstadt (DE); Kathrin Meyer-Böhm, Feucht (DE); Angelika Maschke, Regensburg (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/663,427

(22) PCT Filed: Jun. 2, 2008
(86) PCT No.: PCT/EP08/56766

§ 371 Date: Dec. 7, 2009

(30) Foreign Application Priority Data
Jun. 6, 2007 (EP) 07109713.3

(51) Int. Cl. A61K 31/522; A61K 31/192; A61K 31/4709

(52) U.S. Cl. 514/263.3; 514/568; 514/313

(57) ABSTRACT

The present invention relates to pharmaceutical formulations for the production of chewable and suckable tablets, comprising agglomerates based on sugar or sugar alcohols and disintegrant and water-insoluble polymers in combination with a viscosity-increasing/gel-forming polymer, and the corresponding chewable and suckable tablets.

The present invention relates to pharmaceutical formulations for the production of chewable and suckable tablets, comprising agglomerates based on sugar or sugar alcohols and disintegrant and water-insoluble polymers in combination with a viscosity-increasing/gel-forming polymer, and the corresponding chewable and suckable tablets.

Chewable and suckable tablets are becoming increasingly important for the oral administration of medicinal substances. Such tablets must be easily chewable, have a pleasant taste and must not leave behind a gritty sensation. Furthermore they should be easy to produce, with direct tableting having considerable advantages over wet granulation, and should have high mechanical strength so that they withstand packaging procedures, transport and also pressing out from packaging without damage.

The products and processes described to date do not meet these requirements or do so only very inadequately.

WO 2003/051338 describes a directly tablettable and readily compressible excipient formulation which comprises mannitol and sorbitol. First, an excipient premix is prepared by dissolution of mannitol and sorbitol in water and subsequent spray drying (customary spray drying and SBD method). Mannitol may also be added to this coprocessed mixture. Tablets which additionally comprise disintegrant, glidant, pigment and an active ingredient are said to disintegrate within 60 seconds in the oral cavity.

US 2002/0071864 A1 describes a tablet which disintegrates within 60 seconds in the oral cavity and is mainly formulated from a physical mixture of spray-dried mannitol and a coarse-particle crosslinked polyvinylpyrrolidone and a limited selection of active ingredients. These tablets have a hardness of about 40 N and produce an unpleasant, gritty mouthfeel.

According to U.S. Pat. No. 6,696,085 B2 a methacrylic acid copolymer of type C is to be used as a disintegrant. The methacrylic acid copolymer of type C is a polymer which is resistant to gastric fluid and insoluble in the acidic pH range but water-soluble in the pH range of 7 as is present in the oral cavity. In addition to low hardness (<20 N), the tablets have high friability (>7%) and have a high proportion in the region of 15% by weight of a coarse-particle disintegrant. They consequently have low mechanical strength and, owing to the high proportion of coarse-particle disintegrant, produce an unpleasant, gritty mouthfeel.

EP 0839526 A2 describes a pharmaceutical dosage form consisting of an active ingredient, erythritol, crystalline cellulose and a disintegrant. Furthermore, mannitol is incorporated and crosslinked polyvinylpyrrolidone is used as a disintegrant, so that a physical mixture forms. The tablets are said to decompose within 60 seconds in the oral cavity.

The application WO 2006/029787 describes a tablet which disintegrates in the mouth within 60 seconds and consists of an active ingredient, a water-soluble polyvinyl alcohol/polyethylene glycol copolymer, sugar/sugar alcohol (mannitol) and disintegrant.

It was an object of the present invention to provide chewable and suckable tablets which have a good taste, leave behind a pleasant mouthfeel and at the same time are readily chewable and suckable but nevertheless are also mechanically very stable.

Accordingly, a pharmaceutical preparation for the production of chewable and suckable tablets was found, comprising

A) agglomerates composed of
a1) 60-97% by weight of at least one sugar or sugar alcohol or mixtures thereof,
a2) 1-25% by weight of a disintegrant,
a3) 1-15% by weight of water-insoluble polymers,
a4) 0-15% by weight of water-soluble polymers, and
a5) 0-15% by weight of further pharmaceutically customary excipients, the total of the components al) to a5) being 100% by weight,
   and

B) viscosity increasing or gel-forming polymers.

The preparations may comprise from 20 to 99, preferably from 40 to 90% by weight of agglomerates A) and from 0.1 to 25, preferably from 1 to 15% by weight of components B).

If desired, it is also possible to add from 0 to 5% by weight of lubricant as component C), from 0.1 to 50% by weight of pharmaceutical active ingredients as component D) and from 0 to 25% by weight of further pharmaceutical excipients as component E). In this case, the total of the amounts of A), B) and, if present, C), D) and E) is 100%.

Furthermore, corresponding chewable and suckable tablets have been found.

The excipient content A) has the following specific composition:

The pharmaceutical preparations comprise, as component al), from 60 to 97% by weight, preferably from 70 to 95% by weight, particularly preferably from 75 to 93% by weight, of a sugar, sugar alcohol or mixtures thereof. Suitable sugars or sugar alcohols are trehalose, mannitol, erythritol, isomalt, maltitol, lactitol, xylitol and sorbitol. The sugar or sugar alcohol components are preferably finely divided, with average particle sizes of from 5 to 100 μm. If desired, the particle sizes can be adjusted by grinding. Preferred particle sizes are from 30 to 50 μm. However, it may also be advisable to use particle sizes smaller than 30 μm. It may likewise be advisable to employ sugars or sugar alcohols which comprise mixtures of fractions differing in particle size, for example mixtures of 30 to 70% by weight of a particle size fraction having an average particle size of <30 μm and 30 to 70% by weight of a particle size fraction having an average particle size of 30 to 50 μm. Mannitol, erythritol or mixtures thereof are preferably employed.

Disiritegrants in amounts of from 1 to 25% by weight, preferably 2 to 15% by weight, particularly preferably 3 to 10% by weight, are employed as component a2). The disintegrants are preferably selected from the group consisting of crosslinked polyvinyl-pyrrolidones, croscarmeHose, sodium carboxymethylstarch and L-hydroxypropyl-cellulose. Croscarmellose means according to the invention the sodium and/or calcium salts of crosslinked carboxymethylcellulose. Preferred L-hydroxypropylcelluloses have 5 to 16% hydroxypropoxy groups. Crosslinked polyvinylpyrrolidones are particularly preferred. Such crosslinked polyvinylpyrrolidones are water-insoluble but non film-forming. The crosslinked polyvinylpyrrolidone may have an average particle size of from 2 to 60 μm, preferably less than 50 μm, particularly preferably less than 30 μm.

Water-insoluble polymers in amounts of from 1 to 15% by weight, preferably from 1 to 10% by weight, are used as component a3). Preferred polymers are those which are insoluble in the pH range from 1 to 14, i.e. have a water insolubility which is pH independent at every pH. However, polymers which are water-insoluble at any pH in the pH range from 6 to 14 are also suitable.

The polymers should be film-forming polymers. In this context, film-forming means that the polymers have a minimum film forming temperature of from −20 to +150° C., preferably from 0 to 100° C., in aqueous dispersion.

Suitable polymers are polyvinyl acetate, ethylcellulose, methyl methacrylatetethyl acrylate copolymers, ethyl acrylate/methyl methacrylate/trimethylammoniumethyl methacrylate terpolymers. Butyl methacrylate/methyl methacrylate/dimethylaminoethyl methacrylate terpolymers.

The acrylate/methacrylate copolymers are described in more detail in the European Pharmacopoeia as Polyacrylate Dispersion 30%, in the USP as Ammonia Methacrylate Copolymer and in JPE as Aminoalkyl Methacrylate Copolymer E. Polyvinyl acetate is used as preferred component c). This may be used as an aqueous dispersion having solids contents of from 10 to 45% by weight. In addition, a preferred polyvinyl acetate is one having a molecular weight of from 100 000 to 1 000 000 daltons, particularly preferably from 200 000 to 800 000 daltons.

Furthermore, the formulations may comprise water-soluble polymers in amounts of from 0 to 15% by weight as component a4). Suitable water-soluble polymers are, for example, polyvinylpyrrolidones or vinylpyrrolidone/vinyl acetate copolymers, polyvinyl alcohol/polyethylene glycol graft copolymers, polyethylene glycols and ethylene glycol/propylene glycol block copolymers,

If desired, taste and appearance of the tablets obtained from the formulations can be further improved by adding pharmaceutically customary excipients (components a5)) in amounts of from 0 to 15% by weight, for example such as acidifiers, buffer substances, sweeteners, flavors, flavor enhancers and colorants. The following substances are particularly suitable here: citric acid, tartaric acid, ascorbic acid, sodium dihydrogen phosphate, cyclamate, saccharin sodium, aspartame, menthol, peppermint flavor, fruit flavors, vanilla flavor, glutamate, riboflavin, beta-carotene, water-soluble colorants and finely divided color lakes.

By adding thickeners, such as high molecular weight polysaccharides, the mouthfeel can be additionally improved by increasing the softness and the sensation of volume.

Furthermore, surfactants may also be added as components a5). Suitable surfactants are, for example, sodium lauryl sulfate, dioctyl sulfosuccinate, alkoxylated sorbitan esters, such as polysorbate 80, polyalkoxylated derivatives of castor oil or hydrogenated castor oil, for example Cremophor® RH 40, alkoxylated fatty acids, alkoxylated hydroxyl fatty acids, alkoxylated fatty alcohols, alkali metal salts of fatty acids and lecithins.

Furthermore, finely divided pigments may also be added for further improvement of the disintegration, because they increase the internal interfaces and hence water can penetrate more rapidly into the tablet. These pigments, such as iron oxides, titanium dioxide, colloidal or precipitated silica, calcium carbonates or calcium phosphates, must of course be very finely divided since otherwise a grainy taste once again results.

The preparations comprise, as components B), viscosity-increasing or gel-forming polymers or mixtures thereof. Viscosity-increasing/gel-forming polymers may be: agar, alginates, for example as sodium alginates, carrageenan, guar, locust bean gum, Lara, aloe, pectins, xanthan, gellan, dextran, curdlan, pullulan, hydroxypropylmethylcellulose (HPMC), hydroxypropylcellulose (HPC), sodium carboxymethylcellulose, hydroxyethylcellulose (HEC), starch, modified starch, chitosan, polyacrylic acid sodium, poloxamers and polyvinyl alcohol. Preferred polymers B are xanthan, alginates, HPMC and HPC.

The preparations according to the invention are conventionally admixed with at least one active ingredient for the production of tablets.

It is possible to employ as active ingredients in principle all active ingredients. The active ingredients mentioned below are preferably employed, particularly preferably in the stated dosages.

Zolmitriptan 2.5 mg, rizatriptan 5 mg, diphenhydramine HCl (taste-masked) 20 mg, brompheniramine 5 mg, chlorpheniramine 5 mg, pseudoephedrine (taste-masked) 30 mg,

paracetamol (taste-masked) 250 mg, ibuprofen (taste-masked) 200 mg, acetylsalicylic acid 250 mg (taste-masked), hyoscyamine sulfate 0.125 mg, mirtazapine 15 mg,

selegeline HCl 1.25 mg, ondansetron 4 mg, olanzapine 5 mg, clonazepam 1 mg, cetirizine hydrochloride 10 mg, desloratadine 5 mg, enalapril maleate 5 mg, domperidone maleate 10 mg, scopolamine 0.25 mg,

oxazepam 15 mg, lorazepam 2.5 mg, clozapine 25 mg, dihydroergotamine mesylate 5 mg,

nicergoline 5 mg, phloroglucinol 80 mg, metopimazine 7.5 mg, triazolam 0.5 mg, protizolam 0.5 mg,

tramadol 50 mg, zolpidem tartrate 5 mg, cisapride 5 mg, risperidone 2 mg, azithromycin 100 mg (taste-masked), roxithromycin 50 mg (taste-masked),

clarithromycin 125 mg (taste-masked), erythromycin estolate 250 mg (taste-masked), apomorphine 20 mg, fentanyl 0.6 mg,

amphotericin 10 mg, hydrotalcid 500 mg, magaldrate 500 mg, magnesium salts 10-500 mg, calcium salts 10-500 mg, echinacea extract 80 mg, aluminum oxide 200 mg, magnesium hydroxide 200 mg, montelukast 5 mg as sodium salt, dequalinium chloride 0.25 mg, cetylpyridinium chloride 1 mg, buprenorphine 0.4-8 mg as HCl, lorazepam 1-5 mg disodium fluorophosphate 50-100 mg, ambroxol HCl 20 mg, benzocaine 10 mg, chiorhexidine 2HCl 5 mg, flurbiprofen 10 mg.

Mixtures of active ingredients can also be employed.

The stated dosages represent the absolute amounts of the respective active ingredient per pharmaceutical form. The concentration of the excipient content and of the active ingredient content in the finished pharmaceutical form depends on the size of the pharmaceutical form. In the case of chewable or suckable tablets usual tablet weights of: 100 mg to 2000 mg.

The active ingredients can also be provided with a conventional taste-masking coating. Suitable polymers for such coatings are: aminoalkylmethacrylate copolymer E (Eudragit E or EPO), polyvinyl alcohols in various formulations (Opadry AMB, Kollicoat Protect), combinations of water-insoluble polymers such as, for example, polyvinyl acetate, poly(meth)acrylates (Eudragit NE 30 D, NM 30D, RL, RS, RD, Kollicoat EMM 30 D), ethylcellulose with water-soluble or water-swellable low or high molecular weight substances (povidone, copovidone, HPMC, HPC, polyethylene glycols, poloxamers, polyethylene glycol-polyvinyl alcohol graft copolymers, sugars, sugar alcohols, organic or inorganic salts), combinations of water-soluble film formers (polyethylene glycol-polyvinyl alcohol graft copolymers, HPMC, polyvinyl alcohols) with fats, waxes, fatty acids and fatty alcohols.

The agglomerates A) can be produced by agglomeration in mixers, fluidized-bed apparatuses or spray towers. Solid starting materials and granulating liquid are first mixed with one another and the moist mixed material is then dried. According to the present invention, the granulating liquid used is an aqueous dispersion of component a3), of the water-insoluble polymer.

In one embodiment of the invention, one or more active ingredients are introduced first together with the sugar or sugar alcohol, disintegrant and, if desired, components a4) and a5) into the fluidized bed.

According to a further embodiment, components al) to a5) are agglomerated in the absence of an active ingredient.

In fluidized-bed agglomeration, an aqueous dispersion of the water-insoluble polymer (component a3)) is sprayed onto a fluidized mixture of sugar or sugar alcohol, disintegrant, if desired, active ingredients and, if desired, further components d) and e), resulting in the agglomeration of the fine particles. The inlet air temperatures are 30 to 100° C., and the outlet air temperatures are 20 to 70° C.

Especially with this agglomeration it is possible to incorporate as further components e) the following excipients:

colorants, sweeteners, flavorings, further disintegrants, carbonates, bicarbonates, acidifiers or further excipients. The use of colorants, in which case it is possible to use inorganic pigments, organic color lakes or water-soluble colorants, leads for example to uniformly colored, rapidly disintegrating tablets. Examples of suitable colorants are riboflavin, beta-carotene, anthocyans, carmine, indigocarmine, orange yellow S, quinoline yellow, indigotine lake, brilliant blue, sunset yellow. These further substances can either be put in solid form into the fluidized bed initial charge or be dissolved or dispersed in the dispersion of components a3). If the dispersion is incompatible with such a substance, the latter can also be sprayed on in solution or as suspension before or after the agglomeration with the dispersion of components a3).

In production in spray towers, the so-called FSD or SBD technology (FSD: fluidized spray drying; SBD: spray bed drying) is preferably used. Here, a solution of the sugar or sugar alcohol in water is first spray-dried and the addition of disintegrants and the spraying in of an aqueous dispersion of the water-insoluble polymer are effected in the lower part of the spray dryer or in a connected fluidized bed, with the result that the particles agglomerate. Fine particles can furthermore be blown again in front of the spray nozzle of the sugar or sugar alcohol solution and additionally agglomerated. A procedure starting from the crystalline form of the sugar or sugar alcohol is also possible in the spray tower, FSD or SBD. The crystalline sugar or sugar alcohol is added at the top of the spray tower or in the recycle stream of fine material. By spraying an aqueous dispersion of the water-insoluble polymer, this crystalline solid is agglomerated in the tower.

It may prove advantageous for the agglomeration process to carry out a multistage spray process. At the beginning, the spray rate is kept low in order to prevent over-moistening of the initially charged product and hence adhesion thereof. With increasing duration of the process, the spray rate can be increased and thus the tendency to agglomerate can be raised. It is also possible to adapt the inlet air flow rate and/or temperature in an appropriate manner during the process. Particularly during the drying phase, it is advantageous to reduce the inlet air flow rate and hence to prevent abrasion of the agglomerates due to a high mechanical stress.

The fineness of the spray droplet of the binder solution or dispersion (adjustable via the atomization gas pressure), the nozzle geometry and the distance from the nozzle to the product bed may be regarded as further adaptation parameters for the agglomerate size. The finer and more uniform the spraying, the finer and more uniform are the resulting agglomerates. The further away the nozzle is from the product bed, the poorer is the agglomeration behavior.

Furthermore, the agglomeration can also take place in a mixer by continuous aggregation with mixing. Such a continuous form of aggregation with mixing is the so-called “Schugi granulation”. There, solid starting materials and the granulating liquid comprising the water-insoluble polymer are thoroughly mixed with one another in a continuously operating vertically arranged high-speed mixer (cf. also M. Bohnet, “Mechanische Verfahrenstechnik”, Wiley VCH Verlag, Weinheim 2004, page 198 et seq.).

According to a particular embodiment, the disintegrant is suspended in the aqueous dispersion of the water-insoluble polymer.

The agglomerates thus obtained have an average particle size of 100-600 μm, preferably 120-500 μm and particularly preferably 140-400 μm. The water-insoluble, film-forming polymer serves as an agglomerating agent for agglomerating the fine sugar or sugar alcohol crystals, the active ingredient particles and the disintegrant and, if present, further excipients to particles.

In a further embodiment of the invention, it is also possible initially to agglomerate the excipient content A) and then for an agglomeration of A) to take place in a further granulation step with one or more active ingredients.

In a variant of this embodiment, the agglomerated excipient content and active ingredient are initially introduced into the fluidized bed and granulated by means of a binder solution. The binder solution comprises as binder in this case a water-soluble polymer selected from the group of components d). It is preferably an aqueous solution. The binder concentration can be from 5 to 40% by weight. Preferred binders are water-soluble polyvinylpyrrolidones having Fikentscher K values of from 10 to 100, in particular K30, vinylpyrrolidone-vinyl acetate copolymers composed of 30 to 70% by weight N-vinylpyrrolidone, preferably 40 to 60% by weight, and 30 to 70% by weight, preferably 40 to 60% by weight, vinyl acetate, also polyvinyl alcohols, and graft copolymers of polyethylene glycol and polyvinyl alcohol.

In a second variant, the agglomerated excipient content is initially introduced into the fluidized bed, and the active ingredient content is incorporated into the binder solution described above.

In a further variant, in which the water-soluble binder (component a4)) is obligatorily already present in the preagglomerated excipient content, preferably in amounts of from 0.5 to 10% by weight, the granulating liquid employed is water without polymeric binder. Other excipients from the group of component a5) can in this case be added to the water used for the granulation. It may further be advisable to add one of said sugars or sugar alcohols to the water.

The apparatuses and process parameters used for all these variants of the embodiment in which the excipient content is initially agglomerated are otherwise the same as described above for the simultaneous agglomeration are excipient content and active ingredient content.

The formulations according to the invention can advantageously be used for the production of chewable and suckable tablets.

For the production of the tablets, the customary processes can be used, direct tableting and roll compacting having particular advantages. Owing to the particular properties of the formulations according to the invention, as a rule only active ingredient, formulation according to the invention and a lubricant are required. The tablet formulation is therefore very simple and very reproducible and the process is easy to validate.

Furthermore, the formulations according to the invention have extremely good flowabilities and compressibilities, which lead to mechanically very stable tablets. The hardness of the tablets produced with the aid of the pharmaceutical formulations according to the invention is >50 N. Frequently, the hardnesses are above 80 N, even with the use of active ingredients which are difficult to compress. The friabilities are <0.2%. There is therefore no damage during customary tablet handling.

The formulations according to the invention are therefore very stable during storage and retain their attractive appearance.

### EXAMPLES

The rapidly disintegrating excipient produced by fluidized-bed agglomeration was mixed with active ingredients and viscosity-increasing/gel-forming polymers and 2.0% by weight lubricant (Mg stearate) in a Turbula mixer for 10 min. These mixtures were then tableted in a fully instrumented eccentric press (Korsch XP1) (30 strokes/min).

TABLE 1
Composition of formulations A to D in % by weight

| | A | B | C | D |
|---|---|---|---|---|
| Mannitol (d_{0.5}: 36 μm) | 85.7 | 81.2 | 76.2 | 77.2 |
| Kollidon CL-SF | 4.8 | 4.4 | 4.2 | 4.4 |
| Kollicoat SR30D (solid) | 4.8 | 4.4 | 4.2 | 4.4 |
| Alginate | 2.5 | — | 5.0 | — |
| Xanthural 75 | — | 5.0 | — | 10.0 |
| Caffeine (fine powder) | — | | 8.4 | |
| Flurbiprofen | — | 3.0 | | 2.0 |
| Dequalinium chloride | 0.2 | | | |
| Magnesium stearate | 2.0 | 2.0 | 2.0 | 2.0 |

A 10 mm punch (biplanar, faceted) was used for tableting the mixtures A-D. The tablets were compressed to a weight of 300 mg and a hardness of 40-50 N.

The tablets of mixture D were compressed using a compressive force of 19 kN to a weight of 1000 mg and a diameter of 16 mm.

The tablets were investigated for hardness (HT-TMB-Cl-12 F tablet tester from Kraemer), disintegration time in phosphate buffer of pH 7.2 (ZT 74 disintegration tester, Erweka) and release rate in gastric fluid (release apparatus, Erweka).

TABLE 2
Tablet properties of formulations A-D

| | |
|---|---|
| Tableting data | Tablet parameters |

| | Compressive force | Hardness | Disintegration time |
|---|---|---|---|
| | [kN] | [N] | [min:s] |
| A | 4.3 | 38 | 10:17 |
| B | 5.8 | 50 | 42:06 |
| C | 5.6 | 52 | 19:14 |
| D | 18.9 | 132 | 100:01 |

1.-20. (canceled)

21. A pharmaceutical formulation for chewable and suckable tablets comprising
A) an agglomerated excipient content composed of
a1) 60-97% by weight of a sugar or sugar alcohol,
a2) 1-25% by weight of a disintegrant, selected from the group consisting of crospovidone, croscarmellose, sodium carboxymethylstarch and L-hydroxypropylcellulose,
a3) 1-15% by weight of a water-insoluble, film-forming polymer,
a4) 0-15% by weight of a water-soluble polymer and
a5) 0-15% by weight of a further pharmaceutically excipient, the total of the components al) to a5) being 100% by weight, and
B) at least one viscosity-increasing or gel-forming polymer.

22. The formulation according to claim 21 comprising a component B) in amounts of from 0.1 to 25% by weight.

23. The formulation according to claim 21, comprising as component B one or more polymers selected from the group consisting of agar, alginate, carrageenan, guar, locust bean gum, tara, aloe, pectins, xanthan, gellan, dextran, curdlan, pullulan, hydroxypropylmethyl-cellulose (HPMC) hydroxypropylcellulose (HPC), sodium carboxymethylcellulose, hydroxyethylcellulose (HEC), starch, modified starch, chitosan, polyacrylic acid sodium, poloxamers and polyvinyl alcohol.

24. The formulation according to claim 21, comprising A) an agglomerated excipient content composed of
a1) 60-97% by weight of a sugar or sugar alcohol,
a2) 1-25% by weight of a disintegrant, selected from the group consisting of crospovidone, croscarmellose, sodium carboxymethylstarch and L-hydroxypropylcellulose,
a3) 1-15% by weight of a water-insoluble, film-forming polymer,
a4) 0-15% by weight of a water-soluble polymer and
a5) 0-15% by weight of a further pharmaceutically excipient, the total of the components al) to a5) being 100% by weight,
B) at least one viscosity-increasing or gel-forming polymer,
C) from 0 to 10% by weight, based on the total amount of all components, of a lubricant, and
D) at least one active pharmaceutical ingredient.

25. The formulation according to claim 24, further comprising a pharmaceutical excipient E) in addition to components A) to D).

26. The formulation according to claim 25, wherein component E comprises a disintegrant.

27. The formulation according to claim 24, comprising lubricant D in amounts of from 0.2 to 5%.

28. The formulation according to claim 24, wherein lubricant D comprises magnesium stearate or stearic acid.

29. The formulation according to claim 21, wherein the sugar alcohol comprises mannitol or erythritol or mixtures thereof.

30. The formulation according to claim 21, comprising a croscarmellose as sodium or calcium salt.

31. The formulation according to claim 21, wherein component a2) comprises an L-hydroxypropylcellulose having 5 to 16% hydroxypropoxy groups.

32. The formulation according to claim 21, wherein component a2) comprises crospovidone.

33. The formulation according to claim 21, wherein the water-insoluble film-forming polymer comprises polyvinyl acetate.

34. The formulation according to claim 21, wherein the water-insoluble film-forming polymer is polyvinyl acetate and is employed in the form of an aqueous dispersion.

35. The formulation according to claim 21, wherein the water-soluble polymer is polyvinylpyrrolidone.

36. The formulation according to claim 21, wherein the further pharmaceutically excipient is an acidifier, a sweetener, a flavor, a flavor enhancer, a colorant, a thickener, a surfactant or a finely divided pigment.

37. The formulation according to claim 21, comprising agglomerates A) composed of
a1) 70-95% by weight of a sugar or sugar alcohol,
a2) 2-15% by weight of a disintegrant,
a3) 1-10% by weight of a water-insoluble, film-forming polymer,
a4) 0-2% by weight of a water-soluble polyvinylpyrrolidone, and
a5) 0-15% by weight of a further pharmaceutically excipient.

38. The formulation according to claim 21, comprising agglomerates A) composed of
a1) 75-95% by weight of mannitol or erythritol or a mixture thereof,
a2) 3-10% by weight of a disintegrant,
a3) 1-10% by weight of a polyvinyl acetate,
a4) 0-2% by weight of a water-soluble polyvinylpyrrolidone, and
a5) 0-15% by weight of a further pharmaceutically excipient.

39. The formulation according to claim 21, wherein component B) comprises alginate, xanthan or hydroxypropylmethylcellulose.

40. A chewable or suckable tablet comprising the pharmaceutical formulation according to claim 21.
